(19) United States
(12) Patent Application Publication
(10) Pub. No.: US 2003/0027335 A1
(43) Pub. Date: Feb. 6, 2003

(54) Genome engineering by cell-permeable DNA site-specific recombinases

(76) Inventors: H. Earl Ruley, Nashville, TN (US); Daewoong Jo, Nashville, TN (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/948,193

(22) Filed: Sep. 7, 2001

Related U.S. Application Data
(60) Provisional application No. 60/230,690, filed on Sep. 7, 2000.

(51) Int. Cl. C12N 15/87; C12Q 1/00; C12N 9/22

(52) U.S. Cl. 435/455; 435/199; 435/4

(57) ABSTRACT

The present invention provides polypeptides that contain a site-specific DNA recombinase and a membrane translocation sequence, and nucleic acids that encode such cell-permeable recombinases. The invention also provides methods of stimulating site-specific DNA recombination in cells and in animals using the cell-permeable site-specific DNA recombinases of the invention. Also provided are methods of determining the efficiency of protein transduction into cells; methods of detecting whether site-specific DNA recombination has occurred within a cell; methods of identifying compounds that modulate nuclear metabolism or protein trafficking, uptake, and/or excretion; and methods of identifying peptides that act as membrane translocation signals or that act as nuclear translocation signals or other types of protein targeting signals.

### CROSS-REFERENCE TO RELATED APPLICATIONS

This application claims benefit of priority to U.S. Ser. No. 60/230,690, filed Sep. 7, 2000.

### STATEMENT OF FEDERALLY SPONSORED RESEARCH

This invention was made with government support under Grant No. R01RR13166 awarded by the Public Health Service, National Institutes of Health. The government has certain rights in the invention.

### FIELD OF THE INVENTION

This invention relates generally to methods that involve manipulating (e.g., deleting, inverting, replacing, or translocating) DNA segments using cell-permeable sequence-specific DNA recombinases such as Cre recombinase.

### BACKGROUND OF THE INVENTION

DNA sequence-specific recombinases are widely used as tools for artificially manipulating the genomes of mammalian and non-mammalian organisms. The most widely used recombinase, Cre, was originally isolated from the Escherichia coli bacteriophage P1. Cre functions during P1 phage replication by cleaving the replicating P1 phage DNA at specific sites of defined sequence, known as loxP sites. Each loxP site consists of two 13 base pair inverted repeats, separated by an 8 base pair asymmetric spacer. The relatively long length of its target recognition sites confers a high level of specificity to Cre.

One common application of the Cre-loxP recombination system is to create conditional gene “knockouts” in animals, such as mice, which allows the study of genes which, if globally inactivated, would have a lethal effect. To create a conditional gene knockout using the Cre-loxP system, a pair of loxP sites is first introduced into the chromosomal DNA of an embryonic stem (ES) cell, such that the loxP sites flank a specific DNA segment of interest (for example, a full or partial coding region of a gene, or a larger segment of chromosomal DNA), after which the ES cell containing the modified (“floxed”) DNA is used to introduce the modification into the germline. An animal containing such a germline modification is mated with an animal that has been genetically engineered to express a Cre-encoding transgene in a tissue-or developmentally-restricted manner. This mating produces progeny in which recombination of the floxed DNA segment occurs only in a specific tissue or at a specific time in development. Similarly, Cre-mediated recombination can be used to regulate gene structure and function in cultured cells.

However, genetic engineering using sequence-specific recombinases such as Cre is frequently hampered by difficulties in expressing the recombinase enzyme in the appropriate cells. Plasmid and viral expression vectors are frequently used, but the efficiency of DNA-mediated gene transfer is low, making it necessary to select recombinant cells from the transfected population. For example, fluorescent markers incorporated into the recombinase or expressed from a separate gene permit transduced cells to be sorted by flow cytometry. While viral vector-mediated gene transfer is more efficient than plasmid-mediated gene transfer, viral vectors may introduce additional viral genes with potential undesirable effects into the genome of the target cell. In addition, transduced recombinase genes may integrate into untargeted regions of the genome, resulting in unwanted, continued expression of the enzyme and undesirable secondary recombination events.

Clearly, there is a need for an approach that allows efficient, temporally-regulated (e.g., transient) delivery of sequence-specific DNA recombinases to cells in which site-specific DNA recombination is desired.

### SUMMARY OF THE INVENTION

Sequence-specific DNA recombinases are commonly used to artificially manipulate the genomes of a broad variety of mammalian and non-mammalian cells. The present invention provides cell-permeable sequence-specific DNA recombinases that can be employed in any laboratory process that relies upon site-specific DNA recombination. The cell-permeable recombinases of the invention enter cells efficiently, yet are present only transiently, thereby increasing the efficiency and precision of genetic engineering techniques that employ sequence-specific recombinases. The methods described herein provide the first successful demonstration of the use of protein transduction to effect the enzymatic conversion of a substrate within a living cell or animal.

In a first aspect, the invention features a method of stimulating site-specific DNA recombination in a cell is genetically engineered to undergo site-specific DNA recombination mediated by a site-specific DNA recombinase, including contacting a cell with a polypeptide that contains a site-specific DNA recombinase and a membrane translocation sequence, thereby stimulating site-specific DNA recombination in the cell.

In one embodiment of the first aspect of the invention, the cell is from an animal, such as a mammal, for example, a human or a non-human mammal, e.g., but not limited to, a rodent (e.g., a mouse or a rat), a cow, a sheep, a goat, a pig, a horse, a dog, or a cat. The cell may also be from an animal such as a fish (e.g., a zebrafish, a fugu fish, a salmon, a trout, or a carp), a bird (e.g., a chicken or a quail), an insect (e.g., a fly such as Drosophila melanogaster), or a worm (e.g., Caenorhabditis elegans). In another embodiment of the first aspect of the invention, the cell is within an animal, such as a mammal, bird, fish, insect, or worm.

In a second aspect, the invention features a method of determining the efficiency of protein transduction into a population of cells, including: a) contacting the population of cells with a polypeptide containing a site-specific DNA recombinase and a membrane translocation sequence, wherein the population contains cells that are genetically engineered to undergo site-specific recombination mediated by the site-specific DNA recombinase; and b) determining the number of cells or the percentage of cells in the population that undergo site-specific recombination, thereby determining the efficiency of protein transduction into the population of cells.

In a third aspect, the invention features a method of stimulating site-specific DNA recombination in an animal, including administering a polypeptide that contains a site-specific DNA recombinase and a membrane translocation sequence to an animal containing a cell that is genetically engineered to undergo site-specific recombination mediated by the site-specific DNA recombinase, thereby stimulating site-specific DNA recombination in the animal. In one embodiment of the third aspect of the invention, the animal is a mammal, such as a human or non-human mammal, e.g., but not limited to, a rodent (e.g., a mouse or a rat), a cow, a sheep, a goat, a pig, a horse, a dog, or a cat. The cell may also be from an animal such as a fish (e.g., a zebrafish, a fugu fish, a salmon, a trout, or a carp), a bird (e.g., a chicken or a quail), an insect (e.g., a fly such as Drosophila melanogaster), or a worm (e.g., Caenorhabditis elegans).

In a fourth aspect, the invention features a method of detecting whether site-specific DNA recombination has occurred within a cell, including: a) contacting the cell with a polypeptide containing a site-specific DNA recombinase and a membrane translocation sequence, wherein the cell is genetically engineered to express a reporter gene or a selectable marker gene only after undergoing site-specific recombination mediated by the site-specific DNA recombinase; and b) determining whether the reporter gene or the selectable marker gene is expressed in the cell, whereby expression of the reporter gene or the selectable marker gene indicates that site-specific DNA recombination has occurred within the cell, and whereby lack of expression of the reporter gene or the selectable marker gene indicates that site-specific DNA recombination has not occurred within the cell.

In a fifth aspect, the invention features a method of detecting whether site-specific DNA recombination has occurred within a cell, including: a) contacting the cell with a polypeptide containing a site-specific DNA recombinase and a membrane translocation sequence, wherein the cell is genetically engineered to express a reporter gene or a selectable marker gene only prior to undergoing site-specific recombination mediated by the site-specific DNA recombinase; and b) determining whether the reporter gene or the selectable marker gene is expressed in the cell, whereby lack of expression of the reporter gene or the selectable marker gene indicates that site-specific DNA recombination has occurred within the cell, and whereby expression of the reporter gene or the selectable marker gene indicates that site-specific DNA recombination has not occurred within the cell.

In a sixth aspect, the invention features a method of identifying a compound that modulates nuclear metabolism in a cell, including: a) contacting a population of cells with the compound, wherein the population contains cells that are genetically engineered to undergo site-specific recombination mediated by a site-specific DNA recombinase; b) contacting the population of cells with a polypeptide containing the site-specific DNA recombinase and a membrane translocation sequence; and c) detecting site-specific recombination mediated by the site-specific DNA recombinase, whereby an increase or decrease in the number of cells that undergo site-specific recombination, compared to the number of cells that undergo site-specific recombination in a population of cells not contacted by the compound, identifies a compound that modulates nuclear metabolism in a cell.

In a seventh aspect, the invention features an isolated polypeptide containing a site-specific DNA recombinase and a membrane translocation sequence. In a preferred embodiment of the seventh aspect of the invention, the isolated polypeptide contains the amino acid sequence set forth in SEQ ID NO: 1.

In an eighth aspect, the invention features an isolated nucleic acid encoding a polypeptide containing a site-specific DNA recombinase and a membrane translocation sequence. In preferred embodiments of the eighth aspect of the invention, the isolated nucleic acid encodes the amino acid sequence set forth in SEQ ID NO: 1, and the isolated nucleic acid contains the nucleotide sequence set forth in SEQ ID NO: 2.

In a ninth aspect, the invention features a method of identifying a peptide that acts as a membrane translocation signal, including: a) contacting a population of cells with a polypeptide that contains the peptide and a site-specific DNA recombinase, wherein the population of cells contains cells that are genetically engineered to undergo site-specific recombination mediated by the site-specific DNA recombinase; and b) detecting site-specific DNA recombination mediated by the site-specific DNA recombinase, whereby an increase in the number of cells that undergo site-specific DNA recombination, compared to the number of cells that undergo site-specific DNA recombination in a population of cells contacted by a polypeptide that contains the recombinase but lacks the peptide, identifies a peptide that behaves as a membrane translocation signal. In a preferred embodiment of the ninth aspect of the invention the polypeptide further contains a nuclear localization signal.

In a tenth aspect, the invention features a method of identifying a peptide that acts as a nuclear localization signal, including: a) contacting a population of cells with a polypeptide that contains the peptide, a site-specific DNA recombinase, and a membrane translocation signal, wherein the population of cells contains cells that are genetically engineered to undergo site-specific recombination mediated by the site-specific DNA recombinase; and b) detecting site-specific DNA recombination mediated by the site-specific DNA recombinase, whereby an increase in the number of cells that undergo site-specific DNA recombination, compared to the number of cells that undergo site-specific DNA recombination in a population of cells contacted by a polypeptide that contains the recombinase and the membrane translocation signal but lacks the peptide, identifies a peptide that behaves as a nuclear localization signal.

In an eleventh aspect, the invention features a method of stimulating site-specific DNA recombination in a cell, including culturing a first cell in a culture vessel with a second cell, wherein the first cell is genetically engineered to undergo site-specific DNA recombination mediated by a site-specific DNA recombinase, and wherein the second cell is genetically engineered to secrete a polypeptide containing a site-specific DNA recombinase and a membrane translocation sequence, wherein the first cell is contacted by the polypeptide secreted by the second cell, thereby stimulating site-specific DNA recombination in the first cell.

In a twelfth aspect, the invention features a method of identifying a compound that modulates the delivery of a polypeptide to a cell or the activity of a polypeptide in a cell, including: a) contacting a population of cells with the compound, wherein the population comprises cells that are genetically engineered to undergo site-specific DNA recombination, b) contacting the population of cells with a polypeptide comprising a site-specific DNA recombinase and a membrane translocation sequence; and c) detecting site-specific recombination mediated by the site-specific DNA site-specific recombinase, whereby an increase or decrease in the number of cells that undergo site-specific recombination, compared to the number of cells that undergo site-specific recombination in a population of cells not contacted by the compound, identifies a compound that modulates the delivery of a polypeptide to a cell or the activity of a polypeptide in a cell.

In a thirteenth aspect, the invention features a method of identifying an amino acid sequence that modulates the delivery of a polypeptide to a cell or the activity of a polypeptide in a cell, including: a) contacting a population of cells with a polypeptide comprising a site-specific DNA recombinase and a membrane translocation sequence and an additional amino acid sequence, wherein the population comprises cells that are genetically engineered to undergo site-specific DN recombination; and b) detecting site-specific recombination mediated by the site-specific DNA site-specific recombinase, whereby an increase or decrease in the number of cells that undergo site-specific recombination induced by the polypeptide comprising the site-specific DNA recombinase, the membrane translocation sequence, and the additional amino acid sequence, compared to the number of cells that undergo site-specific recombination induced by a polypeptide comprising the site-specific DNA recombinase and the membrane translocation sequence and lacking the additional amino acid sequence, identifies an amino acid sequence that modulates the delivery of a polypeptide to a cell or the activity of a polypeptide in a cell.

In any of the above aspects of the invention, the site-specific recombination can result in inversion of a target DNA segment, deletion of a target DNA segment, replacement of a target DNA segment, or translocation of a DNA segment. Furthermore, stimulation of site-specific DNA combination can activate or inactivate expression of a cellular gene.

In any of the above aspects of the invention, the site-specific DNA recombinase can be, e.g., Cre recombinase or Flp recombinase; and/or the polypeptide comprising the recombinase can further contain a nuclear localization sequence, e.g., but not limited to, an SV40 large T antigen nuclear localization sequence.

In any of the above aspects of the invention the polypeptide comprising the site-specific DNA recombinase can further comprise an amino acid sequence that targets the delivery of the polypeptide to a specific cell type; the polypeptide can further comprise an amino acid sequence that enhances the uptake of the polypeptide into the circulation of an animal; the polypeptide can further comprise an amino acid sequence that enhances the delivery of the polypeptide across the blood-brain-barrier; the polypeptide can further comprise an amino acid sequence that targets the polypeptide to a specific cell or tissue type; or the polypeptide can further comprise an amino acid sequence that slows excretion of the polypeptide from the body of an animal.

Additional advantages of the invention will be set forth in part in the description which follows, and in part will be apparent from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Definitions

In this specification and in the claims that follow, reference is made to a number of terms which shall be defined to have the following meanings:

As used in the specification and the appended claims, the singular forms “a,” “an” and “the” include plural referents unless the context clearly dictates otherwise. Thus, for example, “a molecule” can mean a single molecule or more than once molecule.

By “site-specific DNA recombination” or “sequence-specific DNA recombination” is meant the cutting and rejoining of a target DNA molecule that depends upon the recognition of one or more DNA sites of defined nucleotide sequence by a site-specific DNA recombinase, i.e., an enzyme that mediates site-specific recombination (see, for example, Sauer, Methods 14:381-392, 1998). Examples of such site-specific DNA recombinases are Cre recombinase and Flp recombinase (which recognize loxP sites and FRT sites, respectively, as is known in the art). Site-specific DNA recombinases such as Cre mediate both intramolecular (excisive or inversional) and intermolecular (integrative) site-specific recombination between recognition sites (such as loxp sites), depending upon the orientation of the recognition site with respect to one another, as is well known in the art (see, e.g., Sauer, Methods Enzymol. 225:890-900, 1993). For example, two recognition sites in the same orientation result in the excision of the intervening DNA, whereas two recognition sites in the opposite orientation result in the inversion of the intervening DNA.

| | |
|---|---|
| Example of a loxP site | |
| (inverted repeats are underlined): | |
| ATAACTTCGTATAATGTATGCTATACGAAGTTAT | (SEQ ID NO:3) |
| TATTGAAGCATATTACATACGATATGCTTCAATA | (SEQ ID NO:4) |
| Example of a FRT site | |
| (inverted repeats are underlined): | |
| GAAGTTCCTATACTTTCTAGAGAATAGGAACTTC | (SEQ ID NO:5) |
| CTTCAAGGATATGAAAGATCTCTTATCCTTGAAG | (SEQ ID NO:6) |

By “membrane translocation signal” or “membrane translocation sequence” or “MTS” is meant a short peptide sequence, within a larger polypeptide, that contains hydrophobic amino acids that facilitate the transport of the entire polypeptide across the plasma membrane of a cell. For example, the presence of an MTS within a polypeptide to be delivered to a cultured cell via tissue cuture medium (or the presence of an MTS within a polypeptide to be delivered to cell within an animal by injection) facilitates entry of the polypeptide into the cell. Signal peptides that target secreted polypeptides across the plasma membrane (thereby facilitating their exit from a cell), as are well known in the art (see, e.g., Du et al., J. Peptide Res., 51:235-243, 1998), may be employed as MTSs in the polypeptides and methods of the invention, as may any other peptide sequence that confers the property of cell permeability upon a site-specific DNA recombinase, for use in the methods of the invention. Examples of MTSs are provided hereinbelow, although any MTS may be used in the methods and compositions of the invention.

By “nuclear localization signal” or “nuclear localization sequence” or “NLS” is meant an amino acid sequence, typically rich in the basic amino acids lysine and arginine, that targets a polypeptide to the nucleus. Many nuclear localization sequences are known in the art (see, e.g., Christophe et al., Cell Signal 12:337-341, 2000; Wente, Science 288:1374-1377, 2000; and Dingwall and Laskey, Trends Biochem. Sci. 16:478-481, 1991), and any amino acid sequence that functions as an NLS may be used in the methods of the invention. One example of an NLS is the SV40 NLS, which has the amino acid sequence KKKRK (SEQ ID NO: 7).

Peptides that function as MTSs, NLSs, and accessory molecules in the methods and polypeptides of the invention will generally range in size from about four to about fifty amino acids in length, although smaller and/or larger sizes can also be used. For example, such peptides can be between about four (or five) and about thirty amino acids in length, e.g., five, eleven, sixteen, or twenty-seven amino acids in length, or between about ten (or fifteen) and about twenty, about thirty, or more (about thirty-five, about forty, about forty-five, about fifty, or more than fifty) amino acids in length.

By “target DNA segment” is meant a portion of DNA that is flanked by recognition sites (for example, but not limited to, loxP sites or FRT sites) that are recognized by a site-specific DNA recombinase (such as, but not limited to, Cre or Flp) and are capable of undergoing site-specific DNA recombination. Depending upon the orientation of the sites, the target DNA segment may be deleted (e.g., when flanked by recombinase recognition sites in the same orientation as one another); inverted (e.g., when flanked by recombinase recognition sites in the opposite orientation to one another); or replaced by a segment of donor DNA flanked by recombinase recognition sites (preferably, heterospecific recognition sites, such as heterospecific loxP sites, to minimize the chance of secondary recombination events that results in deletion of the newly inserted donor DNA). The target DNA segment may be a segment of DNA that is normally present on a chromosome, but has been engineered such that it is flanked with recombinase recognition sites, or it may be a segment of DNA that has been artificially introduced into a chromosome. The target DNA segment may also exist within an isolated DNA molecule, such as a plasmid, a virus, an artificial chromosome, or a linear DNA fragment.

By “non-endogenous” is meant a site-specific DNA recombinase that is not naturally present within the cell into which the recombinase is introduced.

By “modulating” is meant to stimulate or inhibit.

By “nuclear metabolism” is meant any process carried out by the nucleus, including, but not limited to: nuclear import or export, DNA repair, DNA replication, transcription, or chromatin remodeling.

By “isolated polypeptide” is meant a polypeptide of the invention (i.e., a cell-permeable recombinase) that has been obtained, for example, by expression of a recombinant nucleic acid encoding the polypeptide (e.g., in a cell or in a cell-free translation system), by extraction from a natural source (e.g., a prokaryotic or eurkaryotic cell), or by chemically synthesizing the polypeptide.

By “isolated nucleic acid” is meant a DNA molecule obtained by a genetic engineering technique, such as those involving DNA cloning or amplification via the polymerase chain reaction (PCR). An isolated nucleic acid may be (but is not limited to), for example, a recombinant DNA molecule that is: incorporated into a vector, such as an autonomously replicating plasmid or virus; or inserted into the genomic DNA of a prokaryote or eukaryote, e.g., as a transgene or as a modified gene or DNA fragment introduced into the genome by homologous recombination or site-specific recombination; or that exists as a separate molecule (e.g., a cDNA or a genomic or cDNA fragment produced by PCR, restriction endonuclease digestion, or chemical or in vitro synthesis). It also includes any recombinant DNA molecule that encodes any naturally- or non-naturally occurring polypeptide. The term “isolated nucleic acid” also refers to RNA, e.g., an mRNA molecule that is encoded by an isolated DNA molecule, or that is chemically synthesized.

By “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell.

By “pharmaceutically acceptable” is meant a material that is not biologically or otherwise undesirable, i.e., the material may be administered to an individual along with a molecule or compound of the invention (e.g., a cell-permeable recombinase) without causing any undesirable biological effects or interacting in a deleterious manner with any of the other components of the pharmaceutical composition in which it is contained.

### BRIEF DESCRIPTION OF THE DRAWINGS

FIG. 1 is a diagrammatic representation of various cell-permeable Cre fusion proteins.

FIG. 2 is a graph showing the in vitro recombination activity of the GST-Cre-MTS fusion protein.

FIGS. 3A-3B are graphs showing recombination of a floxed GFP gene in Tex.loxp.EG cells exposed to His6-NLS-Cre-MTS.

FIG. 3C is a diagram showing a FACS analysis to determine the percentage of GFP-expressing Tex.loxp.EG cells (i.e., cells that have undergone Cre-mediated recombination), after treatment with cell-permeable Cre.

FIG. 3D is a diagram of a Southern blot showing that increasing concentrations of His6-NLS-Cre-MTS increases recombination of the floxed GFP gene in Tex.loxp.EG cells.

FIG. 4A-4B is a pair of diagrams of PCR assays showing the efficiency of cell-permeable Cre-mediated recombination in S4R embryonic stem cells.

FIG. 5 is a diagram showing the chromosomal region in 123 cells containing the floxed Ig heavy chain allele, before (Targeted) and after (Cre-Deleted) exposure to GST-NLS-Cre-MTS.

FIG. 6(A-C) is a series of graphs showing FACS analyses that detect recombination (indicated by β-galactosidase activity) in splenocytes from Rosa 26R mice injected with His6-NLS-Cre-MTS (black-outlined peak) or buffer (grey peak).

FIG. 7 is a diagram showing a 3′ gene trap vector containing loxP sites.

FIG. 8(A-C) is series of diagrams showing segments of DNA that can be recombined into a floxed segment of genomic DNA (top) for various applications of Cre-mediated genetic engineering of a target gene after the gene has been disrupted by insertion of a 3′ gene trap vector (bottom).

### DETAILED DESCRIPTION OF THE INVENTION

DNA sequence-specific recombinases, such as Cre recombinase, are widely used as tools for artificially manipulating mammalian and non-mammalian genomes; however, such approaches are limited by the inefficiency and other difficulties associated with expressing the recombinase in target cells. To address this problem, we asked whether a protein domain with membrane translocating activity could be used to deliver enzymatically active recombinases into cells, both in culture and in living animals. The results described herein represent the first described use of protein transduction to induce the enzymatic conversion of a substrate in living cells or animals.

There are several potential advantages to delivering recombinases as cell-permeable proteins. First, cells are likely to contain much higher levels of enzyme than can be achieved by intracellular recombinase expression driven by a recombinase-encoding vector. Second, the studies described herein show that the process is highly efficient, as a large percentage of the cells acquire the enzyme. Third, recombination can be induced more rapidly, since protein delivery eliminates the lag before transduced genes can express significant levels of protein. Fourth, the enzyme is available only transiently and is cleared from the cells, thus limiting the occurrence of undesired secondary recombination events. Finally, it is possible to control the exact amount of recombinase enzyme that is delivered to the cell.

The cell-permeable recombinases of the invention can be employed in any laboratory process that relies upon sequence-specific DNA recombination, for example: (i) to manipulate mammalian chromosomes (see, e.g., Lewandoski and Martin, Nat. Genet. 17:223-225,1997), (ii) to insert exogenous DNA at specific sites in the genome (see, e.g., Sauer and Henderson, New. Biol. 2:441-449, 1990) (iii) as a reporter of gene and promoter activity (see, e.g., Dias et al., Anal. Biochem. 258:96-102, 1998; and Thorey et al., Mol. Cell. Biol., 18:3081-3088, 1998), (iv) to simplify production of recombinant viral vectors (see, e.g., Hardy et al., J. Virol., 71:1842-1849, 1997; Morsy et al., Proc. Natl. Acad. Sci. USA 95:7866-7871, 1998; and Vanin et al., J. Virol. 71:7820-7826, 1997) and (v) as a means to achieve conditional expression of an otherwise toxic gene (see, e.g., Arai et al., J. Virol. 72:1115-1121, 1998).

Experiments described herein demonstrate the feasibility of delivering biologically active recombinases to a broad variety of mammalian cells in vitro, ex vivo, and in vivo, as high levels of recombination were observed in various types of recombinase-transduced cell lines and primary cells. Moreover, high levels of recombination were observed in various tissues harvested from mice to which recombinase had been administered intravenously or intraperitoneally. Systemic delivery of cell-permeable Cre to mice was remarkably efficient, even crossing the blood-brain barrier. These results indicate that enzymatically active sequence-specific recombinases can be delivered to a wide variety of cell types, including cells within living animals.

In some cases, exposure of cells to 10 μM His6-NLS-Cre-MTS for two hours was sufficient to induce recombination in over 70% of treated cells. This recombination efficiency is as good, if not better, than that observed following gene transfer. Moreover, the ability to induce recombination in non-activated splenocytes and other terminally differentiated cells is particularly significant, since gene transfer methods are typically less efficient in non-proliferating cells.

The ability to induce sequence-specific recombination using cell-permeable recombinases provides many advantages over current methods, and thus has many applications in biomedical research. First, since protein transduction simplifies delivery of recombinases into a wide variety of cell types, it can replace gene transfer for many routine uses. For example, use of cell-permeable Cre facilitates the testing of floxed alleles engineered into ES cells for their ability to undergo Cre-mediated recombination, prior to the introduction of such floxed alleles into the germline of an animal.

Second, the efficiency of recombination following administration of cell-permeable Cre provides a robust system for regulating gene expression in cultured cells. Depending on the configuration of loxP sites, Cre-mediated recombination can be used to activate or inactivate gene expression. This approach allows gene expression to be more tightly regulated than is currently possible in many existing methods. Moreover, by using different sequence-specific recombinases, it is possible to positively or negatively regulate the expression of multiple genes targeted by these different recombinases within the same cell. Moreover, when used in combination with mice containing targeted genes (e.g., genes modified by loxP sites), cell-permeable recombinases permit the regulated ablation or activation of gene expression in differentiated cells both in vivo and ex vivo. This provides an advantage even when recombination can be achieved by expressing Cre under the transcriptional regulation of a tissue-specific promoter, since conditional gene knockouts in mice often have undesirable effects on cell differentiation and/or survival.

Third, cell-permeable recombinases can be used to induce chromosome deletions within a targeted (e.g., floxed) region of the genome to facilitate genetic studies of specific chromosome regions, analogous to those chromosomal deletion studies described in Justice et al. (Methods 13:423-436, 1997); the appropriate cell-permeable recombinase is administered to a mouse having a site that is a target for recombination (e.g., a segment of DNA flanked on each side by a recognition sequence for a site-specific recombinase such as Cre or Flp), and the physiological result of such chromosomal deletion can be studied. Similar approaches can also be used study the effects of chromosomal translocations generated using cell-permeable site-specific DNA recombinases.

For example, a cell-permeable recombinase can be administered to a library of animals (e.g., but not limited to, rodents or other mammals, birds, fish, insects, or worms) each of which includes a recombination target site within a distinct area of the genome, resulting in a distinct chromosomal deletion for each animal in the library. Animals that display the sought-after phenotype (e.g., in a screen for new tumor suppressor genes, increased susceptibility for developing tumors) can be selected, and the responsible gene identified.

Fourth, the use of cell-permeable Cre recombinase provides the first system in which the effects of enzyme concentration and time on a biochemical reaction has been studied in living cells. Thus, Cre-mediated recombination may also be used as a reporter to quantify factors that influence the kinetics of recombination, such as nuclear transport, chromatin structure, or other aspects of nuclear metabolism. We have observed that the efficiency of recombination mediated by cell-permeable Cre can vary according to cell cycle stage (for example, being less efficient in G1 and more efficient in S-phase), suggesting that factors affecting chromatin structure, such as cell cycle stage, DNA replication, transcription, and DNA damage, influence recombination rates. This approach can be used in high-throughput screens to test the relative genotoxicity of potentially hazardous compounds, e.g., pharmaceutical agents, fertilizers, pesticides, and food additives. A compound that induces a change in nuclear metabolism, as indicated by a change in the efficiency of Cre-mediated recombination, can then be further evaluated for its potential genotoxicity, using methods that are well-known in the art. Other cell-permeable site-specific DNA recombinases that would function analogously to Cre in such assays, e.g., Flp, may also be employed in such high-throughput screening approaches.

Fifth, the present invention provides a method to identify and characterize factors that influence the rate of recombination in cells and in animal tissues. Such factors can affect, e.g., the relative efficiency or rate of uptake of polypeptides comprising site-specific DNA recombinases into the circulatory system, the relative efficiency or rate of dissemination of such polypeptides to various tissues of the body, the relative efficiency or rate of clearance of the protein from the body, the relative efficiency or rate of uptake of protein by specific cell types, the trafficking of the protein from the cytoplasm to the nucleus, and factors that affect DNA or chromatin structure and hence the accessibility of the site-specific recombinase to recombination sites (e.g., but not limited to, loxP or FRT sites). Accessory molecules that can be used to modulate recombination by affecting the above factors include peptide/polypeptide sequences that can be included in the recombinase molecules of the invention, as well as non-peptide/non-polypeptide accessory molecules (e.g., synthetic or naturally-occurring compounds) that influence how cells and tissues interact with site-specific DNA recombinases, such as Cre. Factors discovered by virtue of their effects on a site-specific DNA recombinase such as Cre can then be used to modulate the delivery and activity of proteins other than Cre, e.g., therapeutic proteins.

Because cell-permeable Cre provides a stable record of protein transduction in cells and animals that are capable of undergoing Cre-mediated recombination (i.e., cells and animals that have been engineered such that their genomes contain loxP sites), use of cell-permeable Cre (and analogous recombinases, such as Flp) can facilitate the development of protein-based (e.g., membrane-translocatable) therapies for human diseases, by acting as a marker for protein transduction in cell-based assays and in animals used to test such membrane-translocatable medications.

For example, site-specific DNA recombination mediated by Cre, Flp, or other site-specific DNA recombinases provides a reporter to identify and characterize factors and conditions that can influence the trafficking, uptake, excretion, or other activity of recombinant proteins (e.g., but not limited to, therapeutic recombinant proteins) in cells and animals. Such factors can affect the uptake of recombinant proteins into the circulatory system, the dissemination of recombinant proteins to various tissues of the body, the uptake of recombinant proteins by specific cell types, the delivery of recombinant proteins across the blood-brain barrier, and/or the excretion or clearance of recombinant proteins from the cell or body.

Accessory molecules that influence the specificity and/or efficiency with which cells and tissues take up, metabolize, excrete, and/or otherwise interact with therapeutic recombinant proteins include peptide or polypeptide sequences (which can contain naturally occurring and/or modified amino acids) that can be included in the recombinant protein or added to (e.g., by a peptide bond or other covalent or non-covalent bond) the recombinant protein (e.g., to target the protein to a particular cell or tissue type, or to enhance the stability or delay the excretion or clearance of the polypeptide). Accessory molecule with one or more of the above functions can also be non-peptide/non-polypeptide compounds that can be covalently linked or non-covalently linked (e.g., by a salt bridge, hydrogen bond, hydrophobic bond, or by another non-covalent interaction) to a therapeutic recombinant protein.

For example, the blood-brain barrier is a hindrance to efficacious delivery of therapeutic proteins to the brain. To identify an accessory molecule (e.g., a peptide or polypeptide sequence to be included in a therapeutic protein, or other type of accessory molecule to be covalently linked or non-covalently complexed to the therapeutic protein) that can enhance delivery of a therapeutic protein across the blood-brain barrier, the factor can be covalently or non-covalently combined, as appropriate, with a polypeptide of the invention, e.g., comprising a site-specific DNA recombinase, and optionally an MTS and/or a nuclear localization signal, and the combination can be administered to an animal (e.g., intravenously) such that it will be delivered to (and thus have the opportunity to cross) the blood-brain barrier. An increase (relative to a negative control) in site-specific DNA recombination (e.g., as indicated by an increase or decrease in expression of a reporter gene, e.g., a lacZ gene, engineered to undergo site-specific DNA recombination by a site-specific DNA recombinase, e.g., Cre or Flp) within the cells of the brain indicates that the factor can enhance delivery of a protein across the blood-brain barrier. The identified factor can then be further tested with any specific therapeutic protein in the appropriate animal model to confirm that the factor enhances delivery of the specific therapeutic protein across the blood-brain barrier. It will be clear to one of ordinary skill in the art that an appropriate negative control used to test the ability of a accessory peptide or polypeptide sequence for its ability to enhance delivery of a therapeutic protein across the blood-brain barrier will be a recombinase polypeptide identical to the one used to monitor the efficacy of the accessory peptide or polypeptide, which lacks only the accessory sequence being tested. Similarly, to test an accessory molecule other than a peptide or polypeptide, the negative control will be the recombinase polypeptide in the absence of the accessory molecule being tested.

Sixth, recombination induced by cell-permeable Cre (and other such recombinases) can be used as an assay to test the relative membrane-translocating activity or nuclear targeting activity of various peptide sequences, in order to identify peptides that can maximize the membrane translocation or nuclear targeting of an attached moiety (e.g., a peptide or polypeptide, a nucleic acid (such as an aptamer, antisense oligonucleotide, or ribozyme) or other small molecule. This approach can be used to develop peptides that enhance the delivery of a pharmaceutical compound into the cytoplasm or nucleus.

Seventh, assays based on the detection of site-specific DNA recombination mediated by cell-permeable recombinases can also be used to study protein-protein interactions, and to identify molecules that stimulate or inhibit such protein-protein interactions. For example, a fusion protein is generated that includes Cre, an MTS, an NLS, and NFκB (or just the portion of NFκB that mediates its interaction with IκB). When the fusion protein interacts with IκB within a cell (as does NfκB under normal conditions), Cre-mediated recombination is absent or minimal, because the Cre-containing fusion protein is prevented from translocating to the nucleus by its interaction with IκB. However, in the presence of a stimulus that releases the interaction between NfκB and IκB allows translocation of the Cre-containing fusion protein to the nucleus, and recombination is stimulated. Therefore, this approach can be used to identify factors that regulate NFκB-IκB interactions, and therefore, regulate NfκB-mediated inflammation.

Similarly, assays based on the detection of site-specific DNA recombination mediated by cell-permeable recombinases can be used to study nuclear receptor-ligand interactions, and can be used for high-throughput screens to identify receptor agonists and antagonists. Such agonists and antagonists can be used, e.g., as pharmaceutical agents. In one example of a nuclear receptor for which new ligands can be identified using this approach, the estrogen receptor is a ligand-dependent transcription factor that is localized to the cytoplasm in the absence of its ligand. Upon binding estrogen, a conformational change is induced, which unmasks a previously-sequestered NLS, resulting in the translocation of the hormone/receptor complex into the nucleus. The hormone-responsive binding domain of the estrogen receptor is known, and mutated ligand binding domains of the estrogen receptor, which can be induced by the binding of tamoxifen, are known. Cre-estrogen receptor fusions are also known (Vasioukhin et al., Proc. Natl. Acad. Sci. USA 96:8551-8556, 1999 and Indra et al., Nucleic Acids Res. 27:4324-4327, 1999), but not these fusion proteins are not cell-permeable. Drug-discovery assays can be performed in cells or animals by exposing the cells or animals to fusion polypeptides containing a cell-permeable recombinase and the appropriate estrogen receptor (or other nuclear receptor) fragment. The cells or animals are treated with the test compound, and recombination induced by the recombinase can be measured and compared to that induced by tamoxifen. Competition assays can also be performed, to determine whether a compound is a receptor agonist or antagonist.

Eighth, cell-permeable recombinases can also be delivered to target cells by genetically engineering a first cell type to produce and secrete the cell-permeable recombinase of interest. These cells can then be co-cultured with any type of cell that is a target of the recombinase (e.g., an ES cell). This approach eliminates the need for affinity-purification of the recombinase prior to its use, and also provides continuous exposure of the target cells to the recombinase, thereby maximizing recombination efficiency in the target cell population.

Moreover, cells (e.g.,embryonic stem cells or other type of cells, e.g., primary cells isolated from a subject or tissue culture cells) can be engineered to contain a site for intermolecular (integrative) site-specific DNA recombination at a defined site within the genome by Cre, Flp, or other site-specific recombinases. Any desired DNA sequence can then be introduced into the defined genomic site by intermolecular site-specific DNA recombination. Such a site can include any desired promoter, e.g., a strong promoter for high level expression, or a regulatable promoter, e.g., a tissue-specific, temporally regulated, metallothionein, tetracycline, heat-shock, or other regulatable promoter, of which many examples are well known in the art. In this manner, the desired expression pattern of the DNA sequence (e.g., a protein-coding sequence) introduced into the site can be obtained.

This approach can be used to create transgenic animals (e.g., using engineered embryonic stem cells) e.g., for research or for production of a commercially valuable polypeptide. For example., transgenic goats that secrete commercially valuable (e.g., therapeutic) polypeptides into their milk can be generated using this method. Similarly, cells to be employed for human therapies can be generated by this method. For example, a cell that secretes human insulin, e.g., for administration into a diabetic, can be generated by the methods of the invention.

Finally, although the experiments described herein focus upon transduction of cell-permeable Cre recombinase to mediate recombination via loxP site recognition, similar approaches can be employed using other any sequence-specific DNA recombinase, for example, not only Cre, but any other member of the Int recombinase family (Landy, Curr. Opinion. Genet. Dev. 3:699-707, 1993; Esposito and Scocca, Nucleic Acids Res., 18:3605-3614, 1997), e.g., but not limited to, Flp (see, e.g., Rodriguez et al., Nat. Genet. 25:139-140, 2000; Koch et al., Gene 249:135-144, 2000; Sabath and Shim, Biotechniques 28:966-972, 2000; Dymecki, Proc. Natl. Acad. Sci. USA 93:6191-6196, 1996) and Xer (see, e.g., Comet et al., J. Biol. Chem., 272:21927-21931, 1997).

Membrane Translocation Sequences

Many examples of MTSs are known in the art, including, but not limited to, the Kaposi Fibroblast Growth Factor (KFGF; FGF-4) MTS described in Lin et al. (J. Biol. Chem. 270:14255-14258, 1995); the HIV TAT MTS described in Schwartz et al. (Science 285:1569-1572, 1999) or the HIV TAT MTS set forth in SEQ ID NO: 20 (TGRKKRRQRRR); the Antennapedia MTS described in Derossi et al. (J. Biol. Chem. 269:10444-10450, 1994) or the Antennapedia MTS set forth in SEQ ID NO: 21 (RNIKIWFQNRRMKWKK); the VP22 MTS used to deliver heterologous proteins or peptides into cells (Hawiger, Curr. Opin. Chem. Biol., 3:89-94, 1999; Schwartz and Zhang, Curr. Opin. Mol. Ther. 2:162-167, 2000; Schwartze et al., Trends Cell Biol. 10:290-295, 2000; and Rojas et al., Nat. Biotechnol., 16:370-375, 1998); homeodomains, such as those from the Drosophila melanogaster Fushi-tarazu and Engrailed proteins (Han et al., Mol Cells 10:728-732, 2000); cationic peptides, such as those described in Mi et al. (Mol. Ther. 2:339-347, 2000); e.g., a cationic peptide containing eleven arginines (Matsushita et al., J. Neurosci. 21:6000-6007, 2001; RRRRRRRRRRR; SEQ ID NO: 19), or transportan (Pooga et al., Faseb J. 12:67-77, 1998; GWTLNSAGYLLGKINLKALAALAKKIL; SEQ ID NO: 18). All of the foregoing references are herein incorporated by reference in their entirety.

Test Compounds

In general, compounds (e.g., accessory molecules) that modulate the trafficking, uptake, excretion, or other activity of therapeutic proteins, as indicated by the trafficking, uptake, excretion, recombination activity, or other activity of polypeptides comprising a site-specific DNA recombinase may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Albany Molecular Research, Inc. (Albany, N.Y.) and MediChem (Woodridge, Ill.). Alternatively, libraries of natural compounds in the form of bacterial, fungal, plant, and animal extracts are commercially available from a number of sources, including Aquasearch (Kailua-Kona, Hi., USA), Xenova (Slough, UK), InterBioScreen (Moscow, Russia), and PharmaMar (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their trafficking, uptake, excretion, or other activities should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having the desired activity, as described above. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of potential therapeutic value (e.g., for enhancing protein delivery across the blood-brain barrier) may be subsequently analyzed using an appropriate animal model for a disease or condition in which it would be desirable to alter trafficking, uptake, excretion, or other activity of the specific therapeutic protein.

Administration

The recombinases of the invention and compounds identified using any of the methods disclosed herein may be administered to subjects with a pharmaceutically acceptable diluent, carrier, or excipient, in unit dosage form. By “pharmaceutically acceptable” is meant a material that is not biologically or otherwise undesirable, i.e., the material may be administered to an individual along with a polypeptide or compound of the invention without causing any undesirable biological effects or interacting in a deleterious manner with any of the components of the pharmaceutical composition in which it is contained. Conventional pharmaceutical practice may be employed to provide suitable formulations or compositions to administer such compositions to subjects. Any appropriate route of administration may be employed, for example, but not limited to, intravenous, parenteral, transcutaneous, subcutaneous, intramuscular, intracranial, intraorbital, ophthalmic, intraventricular, intracapsular, intraspinal, intracisternal, intraperitoneal, intranasal, intrarectal, intravaginal, aerosol, or oral administration. Therapeutic formulations may be in the form of liquid solutions or suspensions; for oral administration, formulations may be in the form of tablets or capsules; for intranasal formulations, in the form of powders, nasal drops, or aerosols; for intravaginal formulations, vaginal creams, suppositories, or pessaries; for transdermal formulations, in the form of creams or distributed onto patches to be applied to the skin.

Methods well known in the art for making formulations are found in, for example, Remington: The Science and Practice of Pharmacy (19th ed.) ed. A. R. Gennaro, Mack Publishing Company, Easton, Pa. 1995. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The present invention is more particularly described in the following examples which are intended as illustrative only since numerous modifications and variations thereof will be apparent to those of ordinary skill in the art.

### EXAMPLE I

### Generation of Recombinant Cre Fusion Proteins

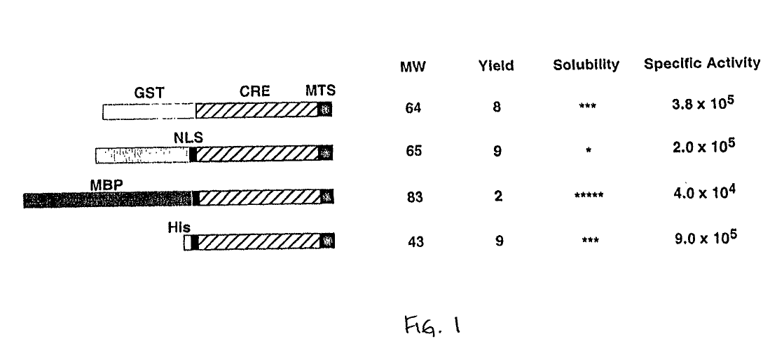

Four recombinant Cre fusion proteins as shown in FIG. 1 were constructed. Each contained the membrane translocation domain (MTS) from the Kaposi fibroblast growth factor (KFGF) (Rojas et al., Nat. Biotechnol. 16:370-375, 1998) positioned at the carboxy-terminus and one of the following affinity tags to facilitate purification: glutathionine-S-transferase (GST; (Novagen Corp, Madison, Wis.), maltose binding protein (MBP; New England Biolabs, Beverly, Mass.), or six histidines (His6; Novagen Corp, Madison, Wis.). In addition, three of the recombinant Cre fusion proteins contained an SV40 T antigen nuclear localization signal (NLS). The molecular weights (MW) of each protein, yield from E. coli cultures expressing the proteins (mg/L), relative solubilities of the purified proteins, and specific activity in vitro (U/mg) are also shown in FIG. 1.

Coomassie blue-stained SDS-polyacrylamide gels containing electrophorectically fractionated lysates from uninduced and IPTG-induced E. coli cultures, and aliquots of recombinant Cre fusion proteins purified by affinity chromatography, showed that all recombinant Cre fusion proteins could be purified to a reasonable degree of homogeneity. Our goal was to characterize a protein with the best combination of yield, solubility and enzymatic activity. For example, addition of the NLS to GST-CRE-MTS enhanced the biological activity of the protein in vivo, but also greatly reduced the yield and solubility of the fusion protein. Replacement of the GST tag with the MBP domain improved solubility but impaired enzymatic activity. The His6-NLS-CRE-MTS had the best combination of yield, solubility and enzyme activity.

GST-CRE-MTS. Cre sequences extending from nt 485 to 1514 (Sternberg, et al., 1986; GenBank X03453; SEQ ID NO: 8) were amplified by PCR with primers A and B. The primers contained a BglII restriction enzyme site which allowed the PCR product to be cloned into the BamHI site of pMTS2 (Rojas et al., Nat. Biotechnol., 16:370-375, 1998). The resulting plasmid expressed GST-Cre-MTS protein under the control of the lacI promoter in E. coli strain BL21. High levels of the fusion protein were expressed 2.5 hours after the addition of 0.6 mM IPTG, and the recombinant protein was purified by glutathione affinity chromatography (as directed by the affinity matrix supplier, Amersham/Pharmacia, Piscataway, N.J.).

| | |
|---|---|
| Primer A: | |
| CCGGAGATCTTAATGTCCAATTTACTGACCGTA | (SEQ ID NO:9) |
| Primer B: | |
| GCCGGAGATCTCATCGCCATCTTCCAGCAGGCG | (SEQ ID NO:10) |

Briefly, bacterial pellets were resuspended in high salt phosphate-buffered saline (PBS; 276 mM NaCl, 2.7 mM KCl, 4.3 mM Na₂HPO₄, 1.4 mM KH₂PO₄) and disrupted by sonication. One-tenth volume of 10% Triton-X 100 was added and samples were centrifuged at 2000 G for 10 min. The clarified lysates were incubated with glutathione beads in high salt PBS overnight at 4° C., washed in high salt PBS, and adsorbed proteins were eluted in buffer containing 1 M NaCl, 100 mM Tris HCl pH 7.4, 20 mM reduced glutathione and 0.1% Triton-X 100 and dialyzed overnight against HEPES-buffered saline (25 mM HEPES, 140 mM NaCl, 7.4 mM Na₂HPO₄, pH 7.4).

GST-NLS-CRE-MTS. The NLS sequence was added to GST-CRE-MTS by PCR amplification of CRE-MTS sequences using a primer (primer C) that contained the five amino acid sequence of the SV40 large T antigen NLS (KKKRK; SEQ ID NO: 7) positioned in-frame with the amino-terminal coding sequence together with primer B used to construct GST-CRE-MTS (see above). Both primers contained a BglII restriction enzyme site which allowed the PCR product to be cloned into the BamHI site of pMTS2 (Rojas et al., Nat. Biotechnol., 16:370-375, 1998).

Primer C: CCGCCGGAGATCTTAATGCCCAAGAAGAAGAGGAAGCTGTCCAATTTACTGACCGTACAC (SEQ ID NO: 11)

Expression and purification of GST-NLS-CRE-MTS was performed as described above for GST-CRE-MTS.

MBP-NLS-CRE-MTS. NLS-CRE-MTS sequences were PCR-amplified from the GST-NLS-CRE-MTS plasmid by using primers D and E. Primers D and E overlap with the coding sequences for NLS and MTS, respectively, and contained sequences, including BglII sites, that allowed the PCR product to be cloned in frame into the BamHI site of the MBP expression vector, pMAL-c2 (New England Biolabs, Beverly, Mass.).

| | |
|---|---|
| Primer D: | |
| CCGCCGAGATCTCCCAAGAAGAAGAGGAAGGTGTCCAATTTACTGACCGTACAC | (SEQ ID NO:12) |
| Primer E: | |
| CCGCCGAGATCTTTAGGGTGCGGCAAGAAGAACAGGGAGAAGAACGGCTGC | (SEQ ID NO:13) |

MBP-NLS-CRE-MTS was purified (Kolb and Siddell, Gene 183:53-60, 1996) from E. coli TB1 cells grown to A₆₀₀ of 0.5 and induced for 5 hrs. with 0.3 mM IPTG. Bacterial pellets were resuspended in lysis buffer (100 mM Tris HCl (pH 7.5), 300 mM NaCl and 1 mM EDTA), disrupted by sonication, and centrifuged (9000×g for 30 min) at 4° C. The clarified supernatants were incubated with amylose resin overnight at 4° C., washed 5 times with lysis buffer, and the fusion proteins were eluted in lysis buffer containing 10 mM maltose and dialyzed overnight against cell culture medium (DMEM or RPMI).

His6-NLS-CRE-MTS. NLS-CRE-MTS sequences were PCR-amplified from the GST-NLS-CRE-MTS plasmid by using primers F and G. Primers F and G overlap with the coding sequences for NLS and MTS, respectively, and contained sequences, including NdeI sites, that allowed the PCR product to be cloned in frame into the NdeI site of the His6 expression vector, pET-28a(+) (Novagen Corp, Madison, Wis.).

| | |
|---|---|
| Primer F: | |
| CCGCCGCATATGCCCAAGAAGAAGAGGAAGGTGTCCAATTTACTGACCGTACAC | (SEQ ID NO:14) |
| Primer G: | |
| CCGCCGCATATGTTAGGGTGCGGCAAGAAGAACAGGGAGAAGAACGGCTGC | (SEQ ID NO:15) |

His6-NLS-CRE-MTS was purified (as directed by the affinity matrix supplier, Qiagen, Valencia, Calif.) from E. coli BL21 cells grown to an A₆₀₀ of 0.8-1.0 and induced for 5 hours with 0.7 mM IPTG. Bacterial pellets were resuspended in lysis buffer (50 mM NaH₂PO₄ (pH 8.0), 300 mM NaCl and 10 mM imidazole), disrupted by sonication, and centrifuged (3000×g for 30 min) at 4° C. The clarified supernatants were incubated with nickel-nitrilotriacetic acid (Ni-NTA) affinity resin for 30 min. at 4° C., washed 3 times with wash buffer (50 mM NaH₂PO₄ (pH 8.0), 300 mM NaCl and 20 mM imidazole), and the fusion proteins were eluted in elution buffer (50 mM NaH₂PO₄ (pH 8.0), 300 mM NaCl and 250 mM imidazole), concentrated in an Amicon ultrafiltration unit and dialyzed overnight against cell culture medium (DMEM or RPMI).

### EXAMPLE II

### Recombinant Cre Proteins are Enzymatically Active in Vitro

The enzymatic activities of recombinant Cre proteins were measured by using the loxP control DNA substrate purchased from Novagen Corp. (Madison, Wis.). The substrate consists of linear plasmid sequences flanked by loxP sites cloned into a λ bacteriophage vector. The plasmid contains an ampicillin resistance gene, and the circular plasmid generated by Cre-mediated recombination, unlike the original substrate, efficiently transforms E. coli to ampicillin resistance.

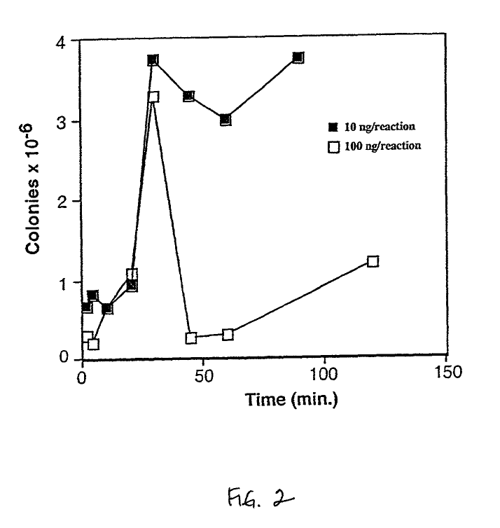

Ten or 100 ng of recombinant GST-Cre-MTS fusion protein was incubated with 200 ng of substrate. At various time intervals, the reaction was stopped by phenol extraction. DNA samples were precipitated in ethanol and used to transform E. coli. Cre-dependent excision of plasmid sequences was monitored by the production of ampicillin-resistant colonies. As illustrated in FIG. 2, the kinetics of Cre-mediated recombination are complicated by the fact that the sequences containing loxP sites continue to recombine. Thus, the initial circular products are further modified, thus reducing their transforming ability. This was particularly evident with higher concentrations of enzyme. Nevertheless, the purified recombinant Cre fusion proteins were highly active, with specific activities ranging from 0.4 to 9.0×10⁵ U/mg protein (FIG. 1), wherein 1 U is the amount of enzyme required to generate 10⁴ ampicillin resistant colonies (equivalent to 2×10⁶ circular molecules) in a 30 minute reaction containing 200 ng DNA substrate in 50 mM Tris-HCl, pH. 7.5, 33 mM NaCl, and 10 mM MgCl₂ in a total volume of 15 μl.

### EXAMPLE III

### Entry of Recombinant Cre Proteins into Mammalian Cells

Uptake of recombinant Cre proteins into mammalian NIH3T3 cells was monitored by confocal fluorescence microscopy. Cells were incubated with serum-free medium alone, or with serum-free medium containing 10 μM GST-Cre-MTS, MBP-NLS-CRE-MTS, or His6-NLS-CRE-MTS for one hour (the use of serum-free medium promotes the entry of cell-permeable Cre into the cells), washed and stained with either anti-GST (rabbit polyclonal, provided by Sheila Timmons, Vanderbilt University), anti-MBP (rabbit polyclonal, New England Biolabs), or anti-Cre (rabbit polyclonal, Novagen) antibodies plus a rhodamine-labeled secondary antibody (goat anti-rabbit IgG; Kirkegaard and Perry, Gaithersburg, Md.). Control cells and cells treated with MBP-NLS-CRE-MTS or His6-NLS-CRE-MTS were also stained with the propidium dye YO-PRO1 (Molecular Probes, Eugene, Oreg.), resulting in green nuclear fluorescence. All of the Cre fusion proteins were efficiently transduced, with 100% of treated cells showing intense staining. GST-Cre-MTS was localized predominantly in the cytoplasm; MBP-NLS-CRE-MTS localized to both cytoplasm and nucleus; and His6-NLS-CRE-MTS was predominantly nuclear. Treatment of cells with increasing concentrations of cell-permeable proteins resulted in increasing protein uptake as assessed by immunostaining.

### EXAMPLE IV

### In Vivo Recombination by Cell-permeable Cre Recombinases

A number of cell types containing single copy floxed genes were used to assess whether transduced Cre protein could elicit recombination in vivo. Tex.loxp.EG is a T lymphoctye line in which Cre-mediated recombination activates the expression of a green fluorescent protein (GFP) gene. Tex.loxp.EG cells were derived by infecting Tex cells (a murine thymoma line derived from p53-deficient mice) with the pBABE.lox.stp.EGFP retrovirus. pBABE.lox.stp.EGFP contains the STOP cassette from pBS302 (Lakso et al., Proc Natl Acad Sci USA 89:6232-6236, 1992) positioned upstream of the enhanced green fluorescent protein gene (EGFP; Clontech, Palo Alto, Calif.) and cloned into the pBABE vector (Morgenstern and Land, Nucleic Acids Res 18:3587-96, 1990). Ectopic retroviral stocks were prepared in the BOSC 23 packaging line (Pear et al., Proc. Natl. Acad. Sci. USA 90:8392-8396, 1993).

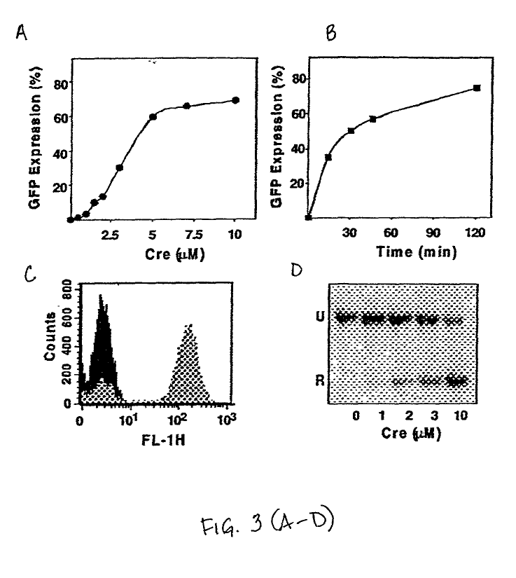

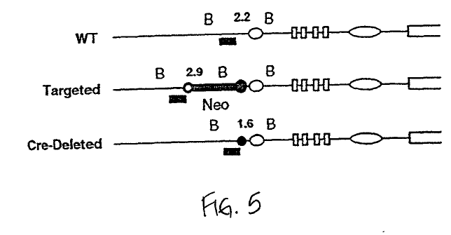

Tex.loxp.EG cells were exposed to His6-NLS-Cre-MTS over a range of protein concentrations for two hours (FIG. 3A) or to 10 μM His6-NLS-Cre-MTS for different lengths of time (FIG. 3B) and the percentage of GFP-expressing cells was assessed by flow cytometry. Cells were washed extensively after exposure to Cre protein and cultured for 24 hrs. to allow time for GFP gene expression. Treatment of cells with 4 μM His6-NLS-Cre-MTS for two hours was sufficient to induce recombination in 50% of cells, which increased to 69% of cells following exposure to 10 μM His6-NLS-Cre-MTS (FIG. 3A). Recombination was also observed in 50% of cells exposed for 30 min. to 10 μM His6-NLS-Cre-MTS, and increased to 75% of cells after 2 hours of treatment with 10 μM His6-NLS-Cre-MTS (FIG. 5B) and to 82% following three consecutive 2-hour treatments with 10 μM His6-NLS-Cre-MTS (FIG. 3C).

Southern blot analysis (FIG. 3D) showed increased conversion of the floxed gene (upper band) to the recombination product (lower band) following exposure to increasing concentrations of His6-NLS-Cre-MTS, confirming that expression of the GFP reporter gene (%GFP) accurately reflected the extent of template recombination. Cre is known to function as a tetramer, consistent with the observed sigmoidal relationship between enzyme concentration and recombination (see FIG. 3A).

S4R embryonic stem (ES) cells contain a single floxed sulfonylurea receptor gene. Cre-mediated recombination generates a unique template that can be specifically amplified by PCR. Specifically, primers (5′-CAATTCCTCAACTGAGGCTCTTAA-3′ (SEQ ID NO: 16) and 5′-GCTTGAAGTTCCTATCCGAAGTTCC-3′ (SEQ ID NO: 17)) complementary to the S4R locus were used to amplify a 351 nucleotide fragment generated by Cre-mediated recombination. PCR reactions (100 ng genomic DNA, 0.2 μM each primer, 0.2 mM each dNTP, 1.5 mM MgCl₂, 1×GeneAmp Gold PCR buffer (Perkin Elmer, Foster City, Calif.) and 2.5 U AmpliTaq gold (Perkin Elmer; Foster City, Calif.) employed 40 cycles of denaturation (94° C.), primer annealing (60° C.), and primer extension (72° C.) for 1 minute each.

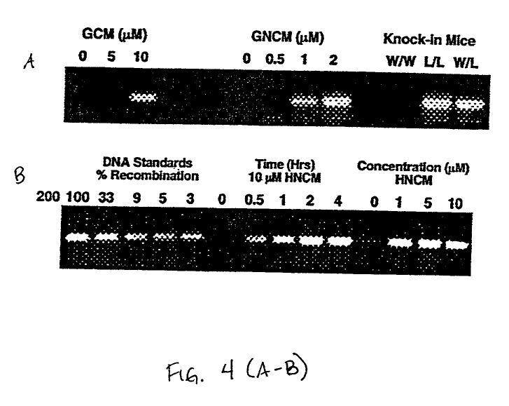

In the experiment represented by FIG. 4A, S4R cells were exposed to the indicated concentrations of GST-Cre-MTS (GCM) or GST-NLS-Cre-MTS (GCNM). DNA from wild type (W/W) mice or mice containing either one (W/L) or two (L/L) deleted alleles was analyzed for comparison. In the experiment represented by FIG. 4B, cells were exposed either to 10 μM His6-NLS-Cre-MTS for different amounts of time or to different concentrations of His6-NLS-Cre-MTS for 4 hrs. Recombination standards were made by diluting DNA with a single deleted allele (100%) with different amounts of wild type DNA.

GST-Cre-MTS induced detectable levels of recombination, but only at the highest concentration (10 μM) of protein tested. By contrast, GST-NLS-Cre-MTS was approximately 10 times more active than GST-Cre-MTS in vivo, even though the protein was slightly less active in vitro and was substantially less soluble (see FIG. 1), suggesting that the presence of the nuclear localization signal in GST-NLS-Cre-MTS is responsible for the increased activity, by virtue of its more efficient targeting of the protein to the nucleus. His6-NLS-CRE-MTS, which also contains a nuclear localization signal, was highly active as well. As compared to DNA standards, exposure of cells to 5-10 μM Cre for two hours was sufficient to induce recombination in 33-100% of templates.

123 is mouse ES cell line containing a single floxed allele of the μ immunoglobulin heavy chain locus. Excision of sequences between the loxP sites converts a 2.9 kilobase BamHI restriction fragment to a 1.6 kB fragment. In the experiment represented in FIG. 5, 123 cells were treated with 10 μM GST-NLS-CRE-MTS for 0, 2, or 4 hours, after which DNA was extracted, digested with BamHI, and analyzed by Southern blot hybridization. A genomic sequence just 5′ of the leftward loxP site (horizontal bar) was used as a probe. GST-NLS-CRE-MTS induced recombination in approximately 20% of 123 cells.

Rosa 26R (R26R) is a transgenic mouse line in which Cre-mediated recombination activates the expression of a β-galactocidase reporter gene (Soriano, Nat. Genet. 21:70-71, 1999). LacZ expression is blocked by four upstream polyadenylation sites which are flanked by loxP sites. Since the R26R promoter that drives lacZ expression is active in all cell types, the R26R locus provides a universal reporter for Cre-mediated recombination. We tested the ability of cell-permeable Cre to elicit recombination in primary splenocytes explanted from R26R mice. Primary splenocytes, including T and B cells, macrophages, and red blood cells were cultured for 24 hours in RPMI medium and treated with serum-free RPMI or with serum-free RPMI containing 10 μM GST-MTS (negative control), 10 μM MBP-NLS-Cre-MTS, or 10 μM His6-NLS-Cre-MTS for two hours. The cells were washed and cultured for three hours in serum-free RPMI to prevent further protein transduction and then cultured in normal media (RPMI plus 10% fetal bovine serum) or in media containing 10 μg/ml lipopolysaccharide (LPS). After 24 hours in culture, the cells were centrifuged onto glass slides and stained with X-Gal. His6-NLS-CRE-MTS induced recombination, as assessed by β-galactosidase expression, in approximately 50% of splenocytes explanted ex vivo. As observed in the previous experiments, MBP-NLS-CRE-MTS was much less active, eliciting β-galactosidase expression in less than 5% of the treated cells. Similar levels of recombination were observed whether or not cells were treated with LPS, a mitogen that stimulates B cell proliferation.

### EXAMPLE V

### Recombination in Mice Treated with Cell-permeable Cre Recombinase

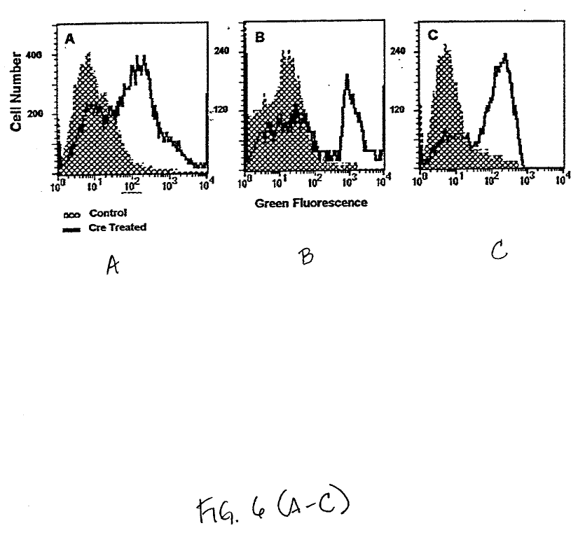

To determine whether cell-permeable Cre recombinase could mediate recombination in intact mice, Rosa26R mice were injected intraperitoneally three times with either 500 μg of His-NLS-Cre-MTS protein (FIG. 6A-6C, black line) or with a buffer (FIG. 6A-6C, grey, filled-in). After three days, β-galactosidase expression in total splenocytes (FIG. 6A), B220-positive cells (i.e., B cells; FIG. 6B), or B220-negative cells (i.e., mostly T cells and macrophages; FIG. 6C) from the Cre recombinase-treated Rosa26R mice was measured by flow cytometry (fluorescence-activated cell sorting; FACS). The enhanced green fluorescence in cells from Cre-treated mice (represented by black-outlined, right-shifted peaks) results from conversion of a fluorescent β-galactosidase substrate, 5-chloromethylfluorescein di-β-D-galactopyranoside (Molecular Probes; Eugene, Oreg.) in cells that have undergone recombination. Therefore, cell-permeable site-specific recombinases such as Cre can be used to induce in vivo recombination in mice genetically engineered to contain recombination target sites for such recombinases.

To further study the ability of cell-permeable Cre to mediate recombination in a broad range of tissue types in vivo, Rosa26R mice were injected intraperitoneally on two consecutive days with 500 micrograms (or on three consecutive days with 25 μg/g body weight) of His-NLS-Cre-MTS in 1 ml of RPMI media or with physiological saline (PBS). Three days later the mice were sacrificed by CO₂ inhalation, and the organs were removed, fixed in 0.25% glutaldehyde for 20 min., and treated with permeabilization buffer (2 mM MgCl₂, 0.01% sodium deoxycholate, and 0.02% NP-40 in PBS) for 20 min. The organs were stained for 24 hrs in 0.2% X-Gal solution as previously described (R. S. Beddington and K. A. Lawson, in: Postimplantation Mammalian Embryos (A. J. Copp and D. L. Cockroft, Eds.), IRL Press, New York, 1990, pp. 267-292) post-fixed in 0.25% glutaraldehyde for 10 min, and examined by dark-field microscopy. Cre-mediated recombination, as evidenced by intense blue staining, was observed in all tissues examined, including the brain. Background staining in some organs (e.g. liver) from control mice is due to low levels of an endogenous beta-galactosidase (C57B 16 mice, which lack the ROSA26R reporter, injected on three consecutive days with 25 μg/g body weight of His-NLS-Cre-MTS also displayed background levels of beta-galactosidase).

Sections through stained liver, brain, and kidney of Rosa 26R mice injected with His-NLS-Cre-MTS showed that lacZ expression was not confined to the surface of the organs or to the vascular system. Moreover, levels of lacZ expression were comparable to those of Rosa26 mice (Zambrowicz, B. P. et al., Proc. Natl. Acad. Sci. U.S.A. 94:3789-3794, 1997) in which lacZ expression is constitutive. Beta-galactosidase was also visualized by immunohistochemical staining of cryosectioned tissues in which beta-galactosidase immune complexes were stained brown with a horseradish peroxidase conjugated secondary antibody. Beta-galactosidase expression in Cre-injected animals was highest in regions surrounding blood vessels in brain and liver sections but was more evenly distributed in the kidney, consistent with systemic delivery of Cre via the bloodstream. Similar results were obtained following intravenous injection, into the tail vein, of Cre recombinase into mice (2.5 μg of Cre per gram of body weight, in 100 μl PBS), as well as in mice injected intraperitoneally for five consecutive days. All mice tolerated the recombinant protein with no apparent adverse effects.

The efficiency of recombination was assessed in mice injected I.P. with 25 μg/g of His6-NLS-Cre-MTS daily for one, three, or five days. After five days, splenocytes and thymocytes were analyzed for lacZ expression by flow cytometry, monitoring the conversion of 5-chlormethylfluorescein di-β-D-galactopyranoside (Molecular Probes, Eugene, Oreg.) to a fluorescent product. The percentage of thymocytes and splenocytes undergoing recombination following one, three and five treatments was approximately 14, 36, and 51% (thymocytes) and 17, 34 and 37% (splenocytes), respectively. In a separate experiment, the efficiency of recombination in total splenocytes was measured at 48% and was somewhat lower in B cells (B220 positive) than in non-B cells (B220 negative), i.e., 43% and 62%, respectively.

The above results show that a wide variety of non-proliferating, terminally differentiated cells can be transduced with cell-permeable proteins in vivo and are competent to undergo Cre-mediated recombination soon after exposure to the enzyme.

### EXAMPLE VI

### Genome Engineering with Cell-permeable Cre Recombinase

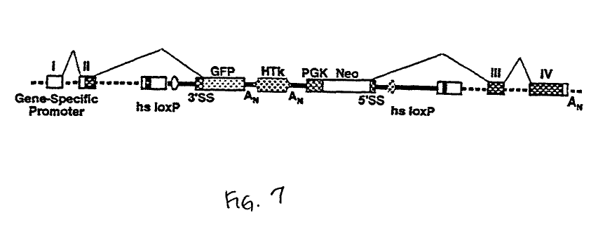

We have developed LNPAT1, a new gene entrapment viral vector that facilitates gene identification and functional analysis (FIG. 7). LNPAT1 functions as a 3′ gene (polyA) trap (Ishida and Leder, Nucleic Acid Res., 27:e35, 1999; Salminen, Dev. Dyn. 212:326-333, 1998; Yoshida et al., Transgenic Res. 4:277-287, 1995; Zambrowicz et al., Nature 392:608-611, 1998) that targets most genes, regardless of whether they are expressed in the target cell. The vector contains a neomycin resistance gene (Neo) under the control of a strong promoter, i.e., the phosphoglycerate kinase (PGK) promoter (Adra et al., Gene 60:65-74, 1987) and ends at a 5′ splice site. The virus inserts the PGKNeo sequence throughout the genome, and selection for G418 resistance gives rise to clones in which Neo sequences can splice to the downstream exons of cellular genes. Since polyadenylation is normally coupled to splicing at the 3′ splice site of the gene's terminal exon, the process traps authentic genes and not cryptic poly(A) sites. 3′ gene trap vectors have been used to target a large number of genes in mouse embryonic stem (ES) cells (Zambrowicz et al., supra). The number and types of targets identified suggest that most genes in the genome can be targeted by this approach.

For example, after selection of ES cells containing gene trap vector insertions, disrupted genes can be identified by sequencing the 3′ cell-encoded portions of the viral-cellular fusion transcripts, which are cloned by 3′ reverse-transcription and amplification of cDNA ends (RACE; Frohman et al., Proc. Natl. Acad. Sci. USA 85:8998-9002, 1988). 3′ RACE is faster, less affected by contaminating plasmid DNA, and requires fewer cells than plasmid rescue; thus, mutant clones can be analyzed at a faster rate. Because the sequence tags are derived from cDNA rather than genomic DNA, the identification of genes disrupted by the virus vector is facilitated (Zambrowicz et al., supra). Since the 3′ RACE products are 3′ anchored cDNAs, they are ideally suited for analysis by high density DNA microarrays (Brown and Botstein, Nat. Genet. 21:33-37, 1999).

In addition to the PGK-Neo gene for selection of cells containing a “trapped” gene, LNPAT1 also contains a green fluorescent protein (GFP; Crameri et al., Nat. Biotechnol. 14:314-319, 1996) reporter gene to detect and monitor expression of the disrupted cellular gene. Since the protein coding capacity of the upstream exons of the trapped gene cannot be predicted in advance, the GFP cassette includes an internal ribosome entry site (IRES), to enhance translation of GFP independent of the translation initiation signals within the endogenous portion of the chimeric mRNA. Transcripts of the trapped gene are processed at a strong poly(A) site located downstream of GFP, thus ablating expression of the trapped cellular gene. The body of the retrovirus (FIG. 7) is opposite from the direction of viral transcription; therefore, the included poly(A) site does not affect synthesis or packaging of vector transcripts. The enhancer sequences normally located in the viral LTRs are deleted to avoid transcriptional effects on adjacent cellular genes.

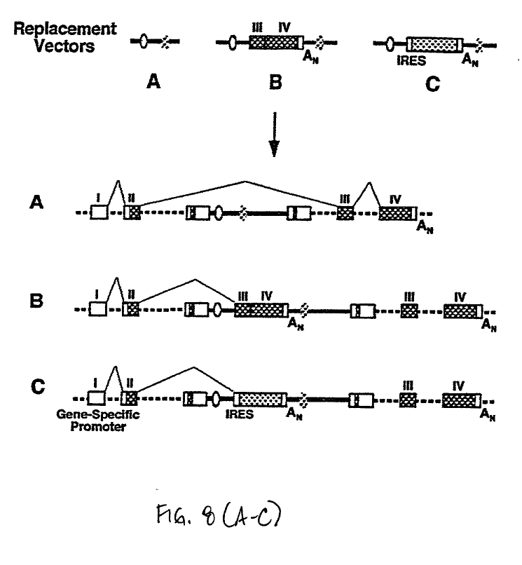

As illustrated in FIG. 8(A-C), heterospecific (hs) loxP sites within the vector allow easy and rapid replacement of the mutagenic vector with other DNA sequences introduced into cells in the presence of Cre recombinase. An inserted herpes simplex virus thymidine kinase (HSV Tk) gene permits selection for loss of vector sequences, as would accompany gene replacement.

The Cre recombinase binds a palindromic target sequence (loxP site) and catalyzes recombination with other loxP sites without the need for additional co-factors or energy source. The reaction is reversible; however mutant loxP sites have been developed that recombine with each other but not with wild type or other mutant loxP sites (i.e., “heterospecific” loxP sites) or that are self-inactivating following recombination. This has been exploited to insert specific sequences in the genome in a position- and orientation specific-manner (Araki et al., Cell Mol. Biol. (Noisey-le-grand) 45:737-750, 1999; Feng et al., J. Mol. Biol. 292:779-785, 1999; Soukharev et al., Nucleic Acids Res., 27:e21, 1999). The process is remarkably efficient, ranging from 10-100% of templates. The use of cell-permeable Cre instead of a transfected Cre expression plasmid streamlines and simplifies the process.

FIG. 8(A-C) illustrates several different applications of cell-permeable Cre-mediated gene replacement, starting with a clone of ES cells containing an endogenous gene disrupted by the gene entrapment vector. The cells can then be used to introduce the modified locus into the germline of an animal (for example, but not limited to, a mouse), either as is, or with further modification. For example, cell-permeable Cre can be used to delete the body of the vector from the disrupted gene (FIG. 8A) by replacement with an empty cassette. Second, cell-permeable Cre can be used to catalyze a reciprocal recombination reaction with a donor DNA molecule that encodes a wild-type, allelic, or mutated form of the disrupted gene (FIG. 8B). The engineered ES cells can then be used to introduce the wild-type, allelic or mutated gene into the germline. For example, animals having a mutated gene that results in a particular disease are useful models for studying that disease. This eliminates a major historical limitation of the gene trap approach, namely, that only large insertion mutations, resulting in loss of function, could be generated, as opposed to more subtle changes, such as allelic variations, point mutations, and small or large deletions.

Finally, a gene disrupted by a 3′ gene trap vector containing recombinase recognition sites (e.g., loxP sites) provides a location for inserting a transgene (FIG. 8C) to be expressed from an upstream cell type-specific or tissue-specific promoter. This approach can be used to characterize a variety of tagged loci in the gene entrapment library, in which the promoter of the targeted gene is expressed at specific times of embryonic development or in specific cell types. The corresponding clones serve as a resource to generate mice that express transgenes under the control of the same regulated promoters. Historically, this has been a significant problem. Moreover, transgene expression in mice generated by pronuclear injection varies greatly from animal to animal, depending on the site of integration and copy number. In contrast, the expression of genes inserted at a specific site in the genome is highly uniform (Soukharev et al., Nucleic Acids Res., 27:e21, 1999).

SEQUENCES

Amino acid sequence of His6-NLS-Cre-MTS (SEQ ID NO:1)
MGSSHHHHHHSSGLVPRGSHMPKKKRKVSNLLTVHQNLPALPVDATSDEVRKNLMDMFRDRQAFSEHTW
KMLLSVCRSWAAWCKLNNRKWFPAEPEDVRDYLLYLQARGLAVKTIQQHLGQLNMLHRRSGLPRPSDSN
AVSLVMRRIRKENVDAGERAKQALAFERTDFDQVRSLMENSDRCQDIRNLAFLGIAYNTLLRIAEIARIRVKDI
SRTDGGRMLIHIGRTKTLVSTAGVEKALSLGVTKLVERWISVSGVADDPNNYLFCRVRKNGVAAPSATSQLS
TRALEGIFEATHRLIYGAKDDSGQRYLAWSGHSARVGAARDMARAGVSIPEIMQAGGWTNVNIVMNYIRNL
DSETGAMVRLLEDGDQIPAAVLLPVLLAAPZ
Nucleotide sequence of His6-NLS-Cre-MTS (SEQ ID NO:2)
ATGGGCAGCAGCCATCATCATCATCATCACAGCAGCGGCCTGGTGCCGCGCGGCAGCCATATGCCCA
AGAAGAAGAGGAA
GGTGTCCAATTTACTGACCGTACACCAAAATTTGCCTGCATTACCGGTCGATGCAACGAGTGATGAGGT
TCGCAAGAACC
TGATGGACATGTTCAGGGATCGCCAGGCGTTTTCTGAGCATACCTGGAAAATGCTTCTGTCCGTTTGCC
GGTCGTGGGCG
GCATGGTGCAAGTTGAATAACCGGAAATGGTTTCCCGCAGAACCTGAAGATGTTCGCGATTATCTTCTA
TATCTTCAGGC
GCGCGGTCTGGCAGTAAAAACTATCCAGCAACATTTGGGCCAGCTAAACATGCTTCATCGTCGGTCCG
GGCTGCCACGAC
CAAGTGACAGCAATGCTGTTTCACTGGTTATGCGGCGGATCCGAAAAGAAAACGTTGATGCCGGTGAA
CGTGCAAAACAG
GCTCTAGCGTTCGAACGCACTGATTTCGACCAGGTTCGTTCACTCATGGAAAATAGCGATCGCTGCCA
GGATATACGTAA
TCTGGCATTTCTGGGGATTGCTTATAACACCCTGTTACGTATAGCCGAAATTGCCAGGATCAGGGTTAA
AGATATCTCAC
GTACTGACGGTGGGAGAATGTTAATCCATATTGGCAGAACGAAAACGCTGGTTAGCACCGCAGGTGTA
GAGAAGGCACTT
AGCCTGGGGGTAACTAAACTGGTCGAGCGATGGATTTCCGTCTCTGGTGTAGCTGATGATCCGAATAA
CTACCTGTTTTG
CCGGGTCAGAAAAAATGGTGTTGCCGCGCCATCTGCCACCAGCCAGCTATCAACTCGCGCCCTGGAA
GGGATTTTTGAAG
CAACTCATCGATTGATTTACGGCGCTAAGGATGACTCTGGTCAGAGATACCTGGCCTGGTCTGGACAC
AGTGCCCGTGTC
GGAGCCGCGCGAGATATGGCCCGCGCTGGAGTTTCAATACCGGAGATCATGCAAGCTGGTGGCTGGA
CCAATGTAAATAT
TGTCATGAACTATATCCGTAACCTGGATAGTGAAACAGGGGCAATGGTGCGCCTGCTGGAAGATGGCG
ATCAGATCCCCG
CAGCCGTTCTTCTCCCTGTTCTTCTTGCCGCACCCTAA

Incorporation by Reference

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of stimulating site-specific DNA recombination in a cell that is genetically engineered to undergo site-specific DNA recombination mediated by a site-specific DNA recombinase, comprising contacting the cell with a polypeptide comprising the site-specific DNA recombinase and a membrane translocation sequence, thereby stimulating site-specific DNA recombination in the cell.

2. A method of stimulating site-specific DNA recombination in an animal, comprising administering a polypeptide comprising a site-specific DNA recombinase and a membrane translocation sequence to an animal comprising a cell that is genetically engineered to undergo site-specific recombination mediated by the site-specific DNA recombinase, thereby stimulating site-specific DNA recombination in the animal.

3. A method of identifying a compound that modulates the delivery of a polypeptide to a cell or the activity of a polypeptide in a cell, comprising:
a) contacting a population of cells with the compound, wherein the population comprises cells that are genetically engineered to under site-specific recombination,
b) contacting the population of cells with a polypeptide comprising a site-specific DNA recombinase and a membrane translocation sequence; and
c) detecting site-specific recombination mediated by the site-specific DNA site-specific recombinase, whereby an increase or decrease in the number of cells that undergo site-specific recombination, compared to the number of cells that undergo site-specific recombination in a population of cells not contacted by the compound, identifies a compound that modulates the delivery of a polypeptide to a cell or the activity of a polypeptide in a cell.

4. A method of identifying an amino acid sequence that modulates the delivery of a polypeptide to a cell or the activity of a polypeptide in a cell, comprising:
a) contacting a population of cells with a polypeptide comprising a site-specific DNA recombinase and a membrane translocation sequence and an additional amino acid sequence, wherein the population comprises cells that are genetically engineered to undergo site-specific DNA recombination; and
b) detecting site-specific recombination mediated by the site-specific DNA site-specific recombinase, whereby an increase or decrease in the number of cells that undergo site-specific recombination induced by the polypeptide comprising the site-specific DNA recombinase, the membrane translocation sequence, and the additional amino acid sequence, compared to the number of cells that undergo site-specific recombination induced by a polypeptide comprising the site-specific DNA recombinase and the membrane translocation sequence and lacking the additional amino acid sequence, identifies an amino acid sequence that modulates the delivery of a polypeptide to a cell or the activity of a polypeptide in a cell.

5. The method of claim 1, 2, 3, or 4, wherein the site-specific DNA recombinase is Cre recombinase.

6. The method of claim 1, 2, 3, or 4, wherein the site-specific DNA recombinase is Flp recombinase.

7. The method of claim 1, 2, 3, or 4, wherein the polypeptide further comprises a nuclear localization sequence.

8. An isolated polypeptide comprising a site-specific DNA recombinase and a membrane translocation sequence.

9. The isolated polypeptide of claim 8, wherein the isolated polypeptide comprises the amino acid sequence set forth in SEQ ID NO: 1.

10. An isolated nucleic acid encoding a polypeptide comprising a site-specific DNA recombinase and a membrane translocation sequence.

11. The isolated nucleic acid of claim 10, wherein the isolated nucleic acid encodes the amino acid sequence set forth in SEQ ID NO: 1.

12. The isolated nucleic acid of claim 10, wherein the isolated nucleic acid comprises the nucleotide sequence set forth in SEQ ID NO: 2.
